(19) United States
(12) Patent Application Publication
(10) Pub. No.: US 2011/0223109 A1
(43) Pub. Date: Sep. 15, 2011

(54) DIAGNOSTIC SUBSTANCE FOR USE IN A METHOD FOR DETERMINING THE AGGRESSIVENESS OF A PROSTATE TUMOR AND DIAGNOSTIC METHOD

(76) Inventors: Jens Fehre, Hausen (DE); Ralf Nanke, Neunkirchen am Brand (DE); Martin Stetter, Munchen (DE)

(21) Appl. No.: 12/671,622

(22) PCT Filed: Jul. 31, 2008
(86) PCT No.: PCT/EP08/60041

§ 371 Date: Feb. 1, 2010

(30) Foreign Application Priority Data
Aug. 6, 2007 (DE) 10 2007 037 008.5

(51) Int. Cl. A61K 49/00

(52) U.S. Cl. 424/9.6; 424/9.1

(57) ABSTRACT

In order to determine the aggressiveness of a prostate tumor, a diagnostic substance is administered to a patient that includes a biomarker provided with a first label that is detectable with a detection device and that specifically binds to a VEGF molecule, and that contains a biomarker that binds specifically to a target molecule that occurs uniformly in the endothelium of blood vessels of healthy tissue and the blood vessels of a prostate tumor, and that is provided with a second label that is detectable with the detection device independently of the first label.

### BACKGROUND OF THE INVENTION

1. Field of the Invention

The present invention concerns a diagnostic substance for application in a method to determine the aggressiveness of a prostate tumor, and such a method.

2. Description of the Prior Art

Every sixth man develops prostate cancer. However, a significant percentage of the tumors are less aggressive and grow so slowly that the patient experiences no complaints during his lifespan. A careful observation is accordingly an important form of therapy in prostate cancer. Assuming this situation it is not sufficient to merely detect the presence of a prostate tumor. Rather, it is of particular importance to receive additional information about its type (and therefore about its aggressiveness) since this is decisive for the therapy to be applied. It has previously been the case that, given suspicion of prostate cancer (aroused by a PSA analysis and rectal digital examination, for instance), biopsies are conducted in order to extract samples from the prostate tissue. The tissue samples are histologically examined and classified into what are known as “Gleason grades” according to their morphology. The less differentiated the tissue, the higher the Gleason grade and the higher the assessed danger or, respectively, aggressiveness of the tumor. This process, what is known as “clinical grading”, has the disadvantage that a biopsy is required for this. Often multiple biopsies with multiple needles are even respectively implemented to increase the precision. Furthermore, it is disadvantageous that the method is based purely on morphological properties of the tissue. Since a biopsy can always miss aggressive tissue, the sensitivity of the method is also limited. Since precise molecular characteristics of the tissue that are responsible for the degree of the aggressiveness are not taken into account, the selectiveness of the method is also limited. Therefore, for many tumors an (often unnecessary) treatment is implemented for safety reasons. Significant stresses of a physical and psychological nature arise for the patient both due to the often repeated biopsies and due to possibly unnecessary treatments.

### SUMMERY OF THE INVENTION

An object of the invention to specify a diagnostic substance for application in a method to determine the aggressiveness of prostate tumors and a corresponding method with which the degree of the tumor aggressiveness can be determined reliably but in manner agreeable to the patient.

A diagnostic substance according to the invention contains a biomarker that is provided with a first label detectable with a detection device and that specifically binds to a VEGF molecule. The invention thereby proceeds from findings concerning the molecular characteristics of prostate cancer tissue. It has been established that the transcription factors Id-1 and Id-2 are more strongly active (and therefore present in higher concentration in the tumor cells) the more aggressive that the tumor is, thus the higher its Gleason grade (Coppe, Itahana et al., Clin. Cancer Res. 10 (2004)). A transcription factor (also called a trans-element) is a protein that is important for the initiation of the RNA polymerase in the transcription. The cited transcription factors (in particular Id-1) are of central importance to the tumorigenic process and tumor dissemination (Wong, Wang et al., Acta Histochem. Cytochem. 37 (2004)). In particular, the high expression of these molecules represents a functional characteristic of aggressive tumors and is therefore a reliable indicator of their aggressiveness as an epiphenomenon, for example the morphological development of tumor tissue. However, Id-1 and Id-2 operate in the intracellular range and therefore are difficult to detect from the bloodstream. They are therefore only less suitable as target molecules that can be detected by biomarkers supplied via the bloodstream.

It is now known that Id-1 drives angiogenesis in prostate cancer, thus the new formation of blood vessels in cancer tissue, wherein this occurs via activation of VEGF (vascular endothelial growth factor) molecules (Ling, Tracy et al., Carcinogenesis 26 (2005)). Since Id-1 exerts its effect by controlling the protein production machinery, it is to be assumed that the amount or density of VEGF is proportional to the amount of Id-1. Thus the quantity or density of VEGF molecules in the area of newly formed blood vessels in cancer tissue is an indicator or, respectively, a measure of the aggressiveness of the prostate cancer. According to the invention, biomarkers are accordingly used that bind to VEGF molecules. In the case of an aggressive prostate cancer, a correspondingly high enrichment of this biomarker then results, which can be detected with the aid of a suitable detection device and with labels detectable by this.

In a method according to the invention, a diagnostic agent that contains a biomarker and a first label connected with this and detectable with a detection device is supplied via the bloodstream to the prostate, wherein a biomarker is used that specifically binds to a VEGF molecule of the vascular endothelium. An enrichment of the biomarker in the region of the cancer tissue is measured with the aid of an extracorporally or intracorporally positioned detection device, wherein the detection device generates a signal whose strength is proportional to the number or, respectively, the density of the VEGF molecules present in a tissue region. The statements above with regard to the advantages connected with the diagnostic substance apply to the method as well.

### BRIEF DESCRIPTION OF THE DRAWINGS

FIGS. 1 and 2 illustrate the different formation of VEGF in the blood vessel wall in the case of prostate cancer with a low degree of aggressiveness, and in the case of prostate cancer with a high degree of aggressiveness, respectively.

FIG. 3 schematically illustrates the basic operation of the diagnostic substance and method in accordance with the present invention.

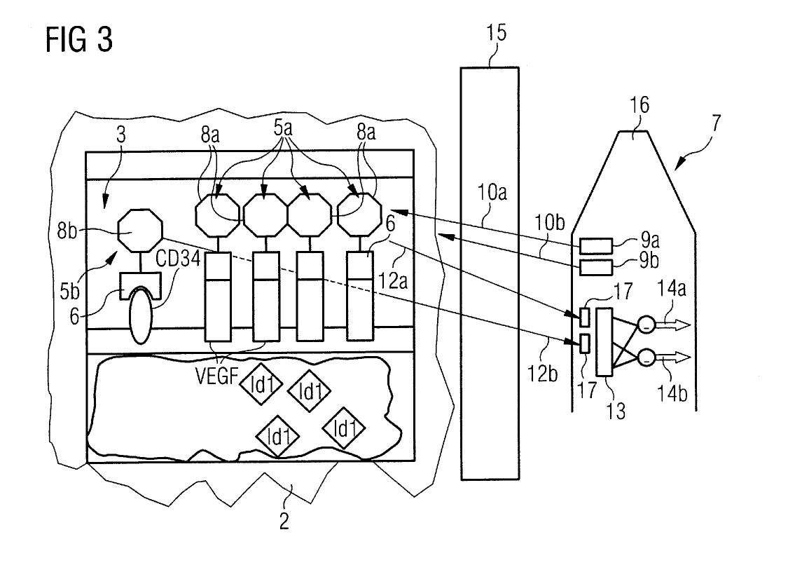

### DESCRIPTION OF THE PREFERRED EMBODIMENTS

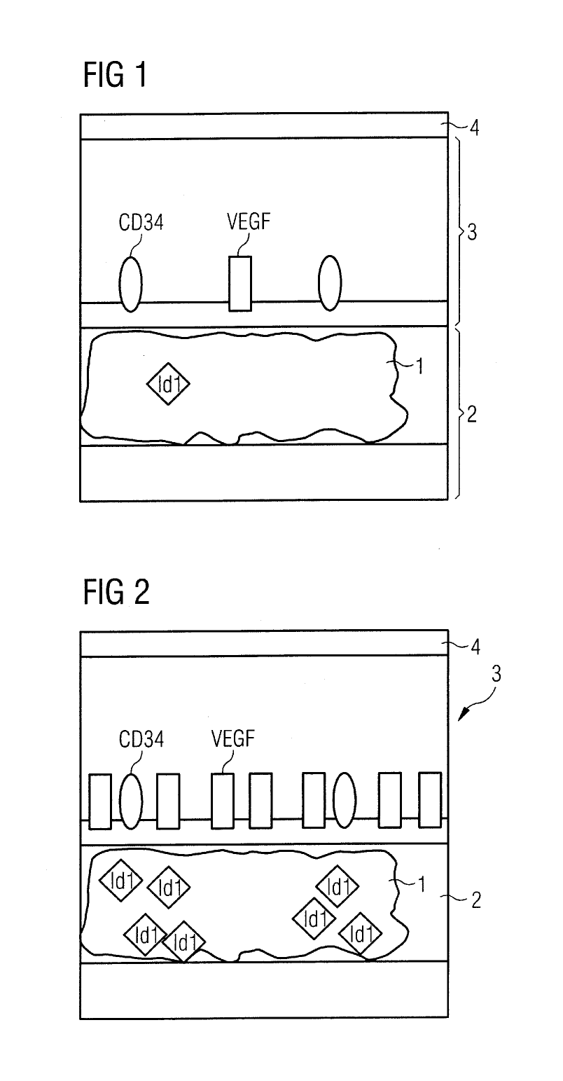

FIG. 1 shows the situation that exists in the case of a tumor 1 of the prostate 2 of low aggressiveness. Only a relatively low concentration of transcription factor Id-1 is present in the prostate tumor 1. The low tumor aggressiveness is linked with a correspondingly low degree of the formation of blood vessels 3. This in turn means that the growth factors VEGF are present with only a low density the endothelium of the blood vessel 4. In contrast to this, the Id-1 concentration and accordingly the number or density of the VEGF molecules in the vascular endothelium is increased in a prostate tumor 1 with high aggressiveness (FIG. 2). The density of VEGF molecules in the vascular endothelium is now established in that a diagnostic substance that contains a biomarker 5a that binds to VEGF molecules is supplied to the prostate 2 via the bloodstream or via blood vessels. The biomarker 5a, like the biomarker described further below, has a binding part that is a molecule or a molecular structure (designated in the following as a coupling molecule 6) and has a label 8 that can be detected with the aid of a detection device 7. Antibodies, aptamers or, respectively, spiegelmers, anticalins and virus particles (in particular M13 phages) come under consideration as coupling molecules 6 suitable for binding to VEGF. Aptamers are short, artificially manufactured RNA or DNA molecules that, like genetic material, are made up of single nucleotides. Spiegelmers are the mirror-reversed equivalents of aptamers. Anticalins are tailored receptor proteins with properties similar to antibodies but are more easily produced than these. With regard to the development of determined, specific binding properties, virus particles (in particular M13 phages) are also of interest. Their protein envelopes can be mutated via targeted biological evolution so that a specific affinity to very specific molecules or molecular structures exists. If a diagnostic substance of the type described above are supplied to the prostate, the biomarkers 5a bind to the VEGF molecules of the vascular endothelium of the blood vessels 3. The greater the bound amount of biomarkers 5a, the greater the amount of detectable labels 8 as well. Apart from the biocompatibility, there are practically no limits with regard to the design of a label. The label 8 must only be detectable in a suitable manner with the detection device 7. In a preferred embodiment, for example, the label 8 is a dye absorbing electromagnetic waves, in particular a dye absorbing and fluorescing in the near-infrared, for example the dye indocyanine green absorbing and fluorescing in the longer-wave range. A detection device 7 suitable for detection accordingly has a light source 9a emitting in near-infrared. The light 10a emitted by this is absorbed by the labels 8a of the biomarker 5a, wherein these emit a fluorescent light that is detected by an infrared sensor 13 and transduced into an electrical signal 14a. The advantage of near-infrared is that it very easily penetrates tissue structures (for instance healthy prostate tissue or the rectal wall 15 in the case of a rectally inserted rectal probe 16 containing the infrared sensor 13), i.e. is only slightly attenuated upon passing through tissue. In order to have a reliable index for the tumor aggressiveness, it is not sufficient to determine only a measurement value proportional to the number of labels 8a and to generate a corresponding signal 14a. Namely, it depends on the density of the biomarker 5a or of the labels 8a. It would now be very difficult to determine the areal region at which the biomarkers 5a are immobilized. In the proposed method, an indirect density determination is achieved in that a biomarker 5b whose coupling molecule 6 binds to a target molecule (for example to the molecule CD 34) that is present uniformly or with uniform distribution in the endothelium of blood vessels of healthy tissue and of blood vessels of the prostate tumor 1 is supplied to the prostate 2. The aforementioned coupling molecules (thus for instance antibodies, anticalins and the like) can serve as coupling molecules 6. In a method variant a diagnostic substance that, in addition to the biomarker 5a, additionally contains a biomarker 5b binding to CD34 is supplied to the prostate tumor 1 via the bloodstream. It is now dependent on the biomarkers 5b being able to be differentiated from the biomarkers 5a interacting with VEGF. For this to be possible, the biomarker 5b binding to CD34 is provided with a second label 8b that can be detected with the detection device 7 independent of the first label 8a of the biomarker 5a. In the case of a detection device 7 operating in the near-infrared, this is ensured in that a dye that absorbs and fluoresces in a different wavelength range than the dye of the first label 8a is used as a second label 8b. For example, the dye NIR-1 that absorbs and fluoresces in a shorter wavelength range than indocyanine green can be used for the label 8b of the dye NIR-1. The detection device 7 then contains a second light source 9b whose emitted light 10b is absorbed by the label 8b. Its fluorescent light 12b is detected by the infrared sensor 13. If applicable, filters 17 which—for example—improve the detection of the fluorescent light 12 can be used in the beam paths. A more reliable index for the density of the VEGF molecules in the prostate tumor 1 can now be achieved if the signal 14a correlated with the fluorescent light 12a of the first label 8a is set in relation to the corresponding signal 14b of the second label 8b.

In an additional method variant, an additional biomarker (not shown) is added to the diagnostic substance, wherein this is designed so that it binds to molecules that are specific to inflamed tissue. For example, the molecule ICAM-1 comes under consideration for this purpose. With this method variant it is possible to establish whether an angiogenesis (i.e. an increased formation of blood vessels) has a different, non-malignant cause. The cited markers can be used in different combinations in order to therefore track different diagnostic goals. Naturally, a serial application of diagnostic substances that respectively contain only one type of biomarker is also conceivable.

In addition to the labels cited above, microbubbles can also be used for example that can be detected with a detection device 7 operating with ultrasound. Another possibility is to use ferromagnetic particles as labels, wherein here a detection device 7 with magnetic sensors or such a device based on MRT can be used.

Although modifications and changes may be suggested by those skilled in the art, it is the intention of the inventors to embody within the patent warranted hereon all changes and modifications as reasonably and properly come within the scope of their contribution to the art.

We claim as our invention:

1-22. (canceled)

23. A diagnostic substance for determining aggressiveness of a prostate tumor, said diagnostic substance comprising:
a first biomarker provided with a first label that is detectable with a detection device and that specifically binds to a VEGF molecule; and
a second biomarker that binds specifically to a target molecule that occurs uniformly in the endothelium of blood vessels of healthy tissue and blood vessels of a prostate tumor, and provided with a second label that is detectable with said detection device independently of said first label.

24. A diagnostic substance as claimed in claim 23 wherein said second biomarker is a biomarker that binds to the CD 34 molecule.

25. A diagnostic substance as claimed in claim 23 comprising a third biomarker that binds specifically to a target molecule that is present in prostate tissue due to inflammation, provided with a third label that is detectable with said detection device independently of said first label and sale second label.

26. A diagnostic substance as claimed in claim 3 wherein said third biomarker binds to ICAM-1.

27. A diagnostic substance as claimed in claim 23 comprising at least one biomarker selected from the group consisting of antibodies, anticalins, aptamers, spiegelmers, and viruses.

28. A diagnostic substance as claimed in claim 23 comprising at least one biomarker containing cultured M13 phage.

29. A diagnostic substance as claimed in claim 23 comprising, as one of said first and second labels, a dye that absorbs electromagnetic waves.

30. A diagnostic substance as claimed in claim 29 wherein said dye absorbs near-infra red electromagnetic waves.

31. A diagnostic substance as claimed in claim 30 wherein said at least one label is a fluorescent dye molecule.

32. A diagnostic substance as claimed in claim 31 wherein said fluorescent dye molecule has a near-infra red absorption spectrum and a near-infra red fluorescence spectrum.

33. A method for non-invasively determining aggressiveness of a prostate tumor, comprising the steps of:
administering a diagnostic substance to a patient, said diagnostic substance comprising a first biomarker provided with a first label that is detectable with a detection device and that specifically binds to a VEGF molecule, and a second biomarker that binds specifically to a target molecule that occurs uniformly in the endothelium of blood vessels of healthy tissue and blood vessels of a prostate tumor, and provided with a second label that is detectable with said detection device independently of said first label; and
detecting said first and second labels with said detection device and generating an extracorporeally available signal with said detection device, from the detected first and second labels, having a signal strength that is proportional to a number or density of VEGF molecules that are present in a tissue region in the patient.

34. A method as claimed in claim 23 comprising administering said diagnostic substance with a biomarker, as said second biomarker, that binds to the CD 34 molecule.

35. A method as claimed in claim 23 comprising administering said diagnostic substance with a third biomarker in said diagnostic substance that binds specifically to a target molecule that is present in prostate tissue due to inflammation, and provided with a third label that is detectable with said detection device independently of said first label and sale second label.

36. A method as claimed in claim 3 comprising administering said diagnostic substance with a biomarker, as said third biomarker, that binds to ICAM-1.

37. A method as claimed in claim 23 comprising administering said diagnostic substance with at least one biomarker selected from the group consisting of antibodies, anticalins, aptamers, spiegelmers, and viruses.

38. A method as claimed in claim 23 comprising administering said diagnostic substance with at least one biomarker containing cultured M13 phage.

39. A method as claimed in claim 23 comprising administering said diagnostic substance with, as one of said first and second labels, a dye that absorbs electromagnetic waves.

40. method as claimed in claim 29 wherein said dye absorbs near-infra red electromagnetic waves.

41. A method as claimed in claim 30 wherein said at least one label is a fluorescent dye molecule.

42. A method as claimed in claim 31 wherein said fluorescent dye molecule has a near-infra red absorption spectrum and a near-infra red fluorescence spectrum.
